(19) United States
(12) Patent Application Publication
(10) Pub. No.: US 2004/0214762 A1
(43) Pub. Date: Oct. 28, 2004

(54) Use of inhibitors of proline endopeptidase to modulate inositol (1,4,5) triphosphate concentration dependent on intracellular signal cascades

(76) Inventors: Hans-Ulrich Demuth, Halle/Saale (DE); Bernd Gerhartz, Lorrach (DE); Ingo Schulz, Halle/Saale (DE)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/486,790

(22) PCT Filed: Aug. 9, 2002
(86) PCT No.: PCT/EP02/08930

Related U.S. Application Data
(60) Provisional application No. 60/312,807, filed on Aug. 16, 2001.

(51) Int. Cl. A61K 38/17; A61K 31/401

(52) U.S. Cl. 514/12; 514/423

(57) ABSTRACT

The present invention discloses a method for the modulation of the enzymatic activity of prolyl endopeptidase in various human tissues and their biological effect on intracellular inositol (1,4,5) triphosphate concentration. The present invention also discloses the potentiation of endogenous neurological and intracellular signaling cascades by inhibition of prolyl endopeptidase activity. This invention further discloses the amplification of substance P mediated stimulation of IP₃ concentration by inhibition of proline endopeptidase activity. The effect of reduced PEP activity on second messenger concentration indicates a novel intracellular function of this peptidase, which has an important impact on cognitive enhancements due to PEP inhibition. Furthermore, this invention discloses the treatment of neuronal disorders such as impaired learning and memory, autoimmune diseases and T-lymphocyte mediated immune disorders and tissue regeneration processes, such as wound healing, which are mediated by activated fibroblasts and/or T-lymphocytes.

### BACKGROUND OF THE INVENTION

1. Field of the Invention

The present invention relates to the function of prolyl endopeptidase in various human tissues and their biological effect on intracellular inositol (1,4,5) triphosphate (IP₃) concentration. The present invention also relates to the potentiation of endogenous neurological and intracellular signaling cascades. This invention further relates to the amplification of substance P mediated stimulation of IP₃ concentration by inhibition of proline endopeptidase activity. The effect of reduced PEP activity on second messenger concentration indicates a novel intracellular function of this peptidase, which has an important impact on cognitive enhancements due to PEP inhibition. Furthermore, this invention relates to the treatment of neuronal disorders such as impaired learning and memory, autoimmune diseases and T-lymphocyte mediated immune disorders and tissue regeneration processes, such as wound healing, which are mediated by activated fibroblasts and/or T-lymphocytes.

2. Background Art

Prolyl endopeptidase (PEP; EC. 3.4.21.26; also called prolyl oligopeptidase) is a serine peptidase characterized by oligopeptidase activity. It is the name giving enzyme of family S9A, prolyl oligopeptidases, in clan SC (1). Enzymes belonging to clan SC are distinct from trypsin- or subtilisin-type serine peptidases by structure and by order of the catalytic triad residues in the primary sequence (2;3). The recently reported three dimensional structure of PEP revealed a two domain organization (4). The catalytic domain displays an α/β hydrolase fold in which the catalytic triad (Ser554, His680, Asp641) is covered by a so-called β propeller domain. Most likely, the propeller domain controls the access of potential substrates to the active site of the enzyme and excludes peptides having more than 30 amino acids.

Despite a profound knowledge on enzymatic and structural properties of PEP, the biological function of this enzyme is far from being fully understood. Highly conserved in mammals, PEP is ubiquitously distributed.

Here we indicate a novel use of PEP inhibition that is related to long term potentiation (LTP) and learning and memory. Using antisense cell lines with reduced PEP expression as well as specific inhibitors, the instant invention demonstrates an inverse correlation between IP₃ concentration and PEP activity. The presented data show an indirect involvement of PEP in second messenger pathways with cross-talk to signal transduction mediated by neuropeptides.

JP 07163367, JP 04066085, JP 05219962 disclose a method for the production of a recombinant prolyl endopeptidase.

JP 06014776 and EP 0522428 disclose the production of prolyl endopeptidase from Aspergillus oryzae FS1-32 (FERM 12193) by cultivation of the microorganism.

JP 07067638 discloses the production of prolyl endopeptidase by cultivation of a bacterial strain belonging to the genus Pseudomonas.

JP 10066570 discloses the production of prolyl oligopeptidase by cultivation of a bacterial strain belonging to the genus Sphingomonas. The isolated prolyl oligopeptidase is claimed to be useful for the production of seasonings and materials for seasonings.

JP 05015314 claims the use of a protease formulation containing a prolyl endopeptidase or carboxypeptidase to carry out the objective removal of bitterness of peptides.

### REFERENCES

1. Handbook of Proteolytic Enzymes (1998). Barrett, A. J., Rawlings, N. D., and Woessner, J. F. London, Academic Press.

2. Goossens, F., De, M., I, Vanhoof, G., Hendriks, D., Vriend, G., and Scharpe, S. (1995) Eur. J. Biochem. 233, 432-441

3. Barrett, A. J. and Rawlings, N. D. (1992) Biol. Chem. Hoppe Seyler 373, 353-360

4. Fulop, V., Bocskei, Z., and Polgar, L. (1998) Cell 94, 161-170

5. Bradford, M. M. (1976) Anal. Biochem. 72:248-54, 248-254

6. Machileidt, W., Nagler, D. K., Assfalg-Machleidt, I., Stubbs, M. T., Fritz, H., and Auerswald, E. A. (1995) FEBS Lett. 361, 185-190

7. Laemmli, U. K. (1970) Nature 227, 680-685

8. Bjerrum, O. J. and Schafner-Nielson, C. (1986) in Electrophoresis 86 (Dunn, M. J., ed) pp. 315-319, VCH, Weinheim

9. Li, J., Wilk, E., and Wilk, S. (1996) J. Neurochem. 66, 2105-2112

10. Johnston, J. A., Jensen, M., Lannfelt, L., Walker, B., and Williams, C. H. (1999) Neurosci. Lett. 277, 33-36

11. Welches, W. R., Brosnihan, K. B., and Ferrario, C. M. (1993) Life Sci. 52, 1461-1480

12. Walter, R., Shlank, H., Glass, J. D., Schwartz, I. L., and Kerenyi, T. D. (1971) Science 173, 827-829

13. Shinoda, M., Miyazaki, A., and Toide, K. (1999) Behav. Brain Res. 99, 17-25

14. Shishido, Y., Furushiro, M., Tanabe, S., Taniguchi, A., Hashimoto, S., Yokokura, T., Shibata, S., Yamamoto, T., and Watanabe, S. (1998) Pharm. Res. 15, 1907-1910

15. Toide, K., Shinoda, M., Fujiwara, T., and Iwamoto, Y. (1997) Pharmacol. Biochem. Behav. 56, 427-434

16. Berger, A. and Schlechter, I. (1976) Biochem. Biophys. Res. Com. 27, 157-162

17. Demuth, H. U., Schienzig, D., Schierhorn, A., Grosche, G., Chapot-Chartier, M. P., and Gripon, J. C. (1993) FEBS Lett. 320, 23-27

### SUMMARY OF THE INVENTION

To investigate its intracellular function, the expression of prolyl endopeptidase was reduced in the astro glioma cell line U343 by antisense technique. Determination of the inositol (1,4,5) triphosphate (IP₃) revealed an inverse correlation between IP₃ concentration and PEP expression in the generated antisense cell lines. Complete suppression of PEP activity by the specific PEP inhibitor, Fmoc-alanyl-pyrroline-2-nitrile (5 μM) induced in U343 wild-type cells an enhanced, however delayed increase of IP₃ concentration, indicating that proteolytic activity of PEP is responsible for the observed effect. Furthermore, the reduced PEP activity was found to amplify substance P mediated stimulation of IP₃ concentration. The effect of reduced PEP activity on second messenger concentration indicates a novel function of this peptidase, resulting in cognitive enhancements due to PEP inhibition. This may be accomplished in accordance with the present invention using especially orally active, low molecular weight inhibitors of prolyl endopeptidase.

### BRIEF DESCRIPTION OF THE DRAWINGS

Further understanding of the present invention may be had by reference to the accompanying drawings wherein:

FIG. 1 shows a agarose gel electrophoresis of the cDNA of the catalytic domain of PEP from human glioma cell line U343 after nested RT-PCR. {Total RNA was prepared from 1×10⁷ U343 cells. The coding region of PEP was amplified by RT-PCR and cDNA of the catalytic domain (amino acid 442-731) was obtained by nested primers (lane 1). Detection of actin mRNA was used as a positive control (lane 2)};

FIG. 2 shows the Western blot analysis of the PEP-expression in established antisense cell lines. {The remaining PEP-activity in each antisense cell line corresponds to the signal intensity in the Western-blot analysis. 1×10⁷ cells from each cell line were extracted and analyzed as described under “Experimental Procedures”. 20 μg of total protein were loaded per lane. Purified recombinant human PEP was used as positive control (75 ng). Western-blot's were probed with PEP specific antibody S449 (1:400) and anti-actin (1:2500) and detected by chemiluminescence.};

FIG. 3 shows the analysis of IP₃ concentration on various U343 cell lines. {A) Reduced PEP activity induces an increased IP₃ concentration in stable transfected cell lines. Human glioma cell line U343 was transfected with a vector (pIRES) containing the coding sequence of the PEP catalytic domain (amino acid 442-731) in antisense direction. The cell line transfected with a vector not harboring an insert (pIRES) was used as a negative control. B) Wild-type U343 cells treated with specific PEP inhibitor, Fmoc-Ala-Pyrr-CN (5 μM) display an increased IP₃ concentration; Data are obtained in quadruplicate (mean±SD) and analyzed using by unpaired t-test (***p<0.001; **p<0.01; *p<0.05; n.s. no significance)};

FIG. 4 shows the time course of PEP activity and IP₃ concentration in U343 cells treated with PEP-inhibitor Fmoc-Ala-Pyrr-CN. {Whereas PEP-activity (▴) was found to be totally inhibited already after 1 min of a single treatment with 5 μM Fmoc-Ala-Pyrr-CN the IP₃ concentration (◯) required 12 hour to reach maximum concentration. Results are presented as mean±standard error from experiments in quadruplicate.};

FIG. 5 shows the detection of neurokinin receptor mRNA's in the human glioma cell line U343. {Total RNA was prepared from 1×10⁷ U343 cells. Expression of NK-R mRNA's was detected with RT-PCR using specific primers; lane1 NK-1R, lane2 NK-2R, lane3 NK-R3.};

FIG. 6 shows the PEP activity measured in various cell compartments of U343 glioma cells. {PEP activity is mainly present in the cytosol of U343 cells. Additionally, small traces of PEP activity was detected in other cell structures and are most likely due to the insufficient separation of compartments. Conditioned media and cells were separated following cell lysis and cell fractionation as described under “Experimental Procedures”. PEP activity was determined in conditioned media (CM), total cell extract (CE), and in enriched fractions of nucleus (P1), vacuoles (P10), cell structural components (P100), and the cytoplasmic proteins (S100) shown as mean±SD of three independent experiments; n.d. not detectable.};

FIG. 7 shows IP₃ concentrations in various U343 cell lines stimulated by substance P. {IP₃ concentrations were measured in U343 wild-type cells with or without incubation in the presence of 5 μM Fmoc-Ala-Pyrr-CN for 12 hours and in cell line as2. Each cell line was stimulated with 1 μM substance P for 5 seconds following IP₃ extraction and measurement. Data (mean±SD) were obtained in quadruplicate and significant analysis was performed by paired t-test (**p<0.01; *p<0.05;).};

FIG. 8 shows the kinetic profile of IP₃ stimulation by substance P in U343 cells. {A) The kinetic profile of IP₃ stimulation by substance P reveals a slight increased, but similar pattern in inhibitor treated (●) and control cells (◯). U343 wild-type cells were treated with 5 μM Fmoc-Ala-Pyrr-CN ( [symbol] ) for 12 hours ahead of the experiment. B) Anti sense cell line 2 from U343 (Δ) display a similar stimulation pattern as wild-type cells; Cells were stimulated with 1 μM substance P and were harvested at different time points to extract IP₃. All time points, presented as the mean±SD are of experiments in quadruplicate.};

FIG. 9 shows the western blot analysis of the PEP-expression in different human cell lines. {The PEP-signals, detected in 6 cultivated human cell lines (1 U343, 2 LN-405; 3 SH-SY5Y; 4 BeWo; 5 U-138-MG; 6 CACO-2) correlates with the PEP-activity, measured in these. No signals at all could be detected in non-cultivated cryo-stocks. Cytosolic supernatants from each cell lines were analyzed. Ten μg (lane 1-3), 20 μg (lane 5) and 40 μg (lane 4, 6) of cytosolic supernatants were loaded per lane. Western-blot was incubated with PEP specific antibody S449 (1:400) and detected by chemiluminescence technique.}; and

FIG. 10 shows the Western blot analysis of the PEP-expression in rat brain. {The PEP-activity, measured in each brain-regions correlates with the signals in the Western-blot analysis. Tissue from each brain-regions (1 cortex, 2 hippocampus, 3 medulla oblongata, 4 cerebellum, 5 thalamus, frontal lobe) were analyzed. Thirty μg of total protein were loaded per lane. Cytosolic U343 cell line supernatant was used as positive control (M, 20 μg/lane). Western-blot was incubated with PEP specific antibody S449 (1:400) and detected by chemiluminescence technique.}

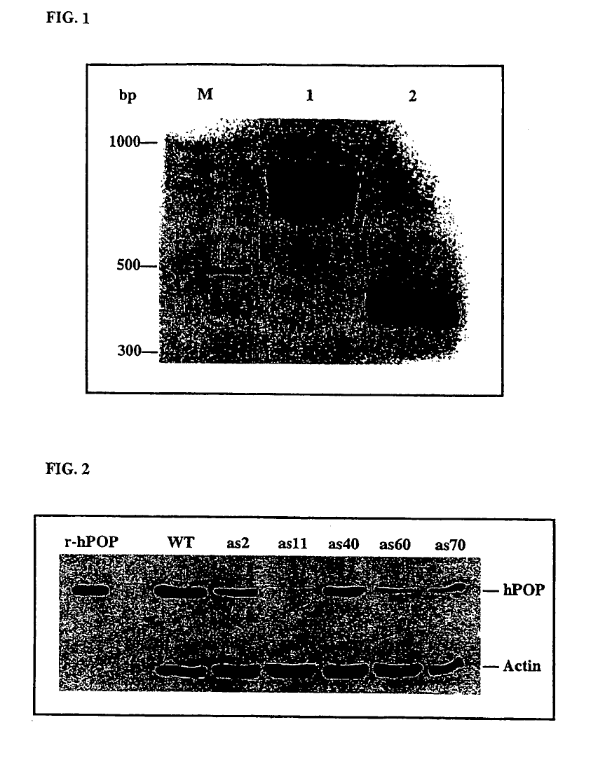

### DETAILED DESCRIPTION OF THE INVENTION

The present invention especially refers to the use of an inhibitor of prolyl endopeptidase for the preparation of a medicament for the modulation of the intracellular level of inositol (1,4,5) triphosphate.

Prolyl endopeptidase (PEP; EC. 3.4.21.26; also called prolyl oligopeptidase) is a serine peptidase characterized by oligopeptidase activity. Due to its peptidase activity, PEP is involved in intracellular signaling and the mediation of intracellular signaling cascades.

Surprisingly, in cell culture experiments we could not determine PEP in the extracellular space, but found PEP mainly intracellularly localized. The present invention shows, that the inhibition of intracellular PEP activity can be used to modulate peptide hormone dependent intracellular inositol (1,4,5) triphosphate (IP₃) concentration. More surprisingly, PEP does not cleave the signaling peptide hormone, because the peptide hormones are extracellularly located and are not accessible to the intracellular located enzyme, but PEP is involved in the intracellular mediation of peptide hormone induced receptor signals. Further, the inhibition of PEP activity does not influence substance P concentration, but enhances the substance P—receptor induced signal of intracellular IP₃ concentration alteration.

First described in 1971 as an oxytocin-inactivating enzyme (12), our knowledge about prolyl endopeptidase has only increased with respect to enzymatic and structural properties, but the physiological function remains unclear.

PEP inhibitors are in general very specific due to the proline residue in P₁-position (Berger and Schlechter nomenclature, (16)) of the substrate. However, two different methods of inhibition have been used. Antisense cell lines expressing PEP in a reduced manner enable investigations on the biological function of non-enzymatic properties of this two-domain protein. Additionally, this technique avoids possible unspecific effects of the reactive group within the inhibitor. Eight stable antisense cell lines have been developed with different amounts of reduced PEP expression. In all cell lines a strong correlation was observed between a reduced PEP expression and the remaining enzyme activity (Table 1). Although differences in cultivation and morphology in these cell lines could be observed, no common change in the phenotype was present. The observed changes seem to be related to the used method of generating antisense cell lines where the antisense encoding DNA has to be inserted into the genome in a random manner. Phenotypic changes in U343 cells were not seen when cells were cultivated in presence of PEP inhibitors.

Antisense oligonucleotides are per definition nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides long, but can be at least 12, 15, 20, 25, 30, 35, 40, 45 or 50 or more nucleotides long. Longer sequences also can be used. Antisense oligonucleotide molecules can be provided in a DNA construct and introduced into a cell as described above to decrease the level of prolyl endopeptidase gene products in the cell.

Antisense oligonucleotides can be deoxyribonucleotides, ribonucleotides, or a combination of both. Oligonucleotides can be synthesized manually or by an automated synthetizer, by covalently linking the 5′end of one nucleotide to the 3′end of another nucleotide with non-phosphodiester internucleotide linkages such as alkylphosphonates, phosphorothioates, phosphorodithioates, alkylphosphonothioates, phorphoramidates, phosphate esters, carbamates, acetamidate, carboxymethyl esters, carbonates, and phosphate triesters. See Brown, Meth. Mol. Biol. 20, 18, 1994; Sonveaux, Meth. Mol. Biol. 26, 1-72, 1994; Uhlmann et al., Chem. Rev. 90, 543-583, 1990.

Modifications of prolyl endopeptidase enzyme gene expression can be obtained by designing antisense oligonucleotides which will form duplexes to the control, 5′, or regulatory regions of the prolyl endopeptidase enzyme gene. Oligonucleotides derived from the transcription initiation site, e.g., between positions 10 and +10 from the start site, are preferred. Similarly, inhibition can be achieved using “triple helix” base pairing methodology. Triple helix pairing is useful because it causes inhibition of the ability of the double helix to open sufficiently for the binding polymerases, transcriptions factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an anti sense oligonucleotide and the complementary sequence of the prolyl endopeptidase enzyme polynucleotide. Antisense oligonucleotides which comprise, for example, 2, 3, 4 or 5 or more stretches of contiguous nucleotides which are precisely complementary to the prolyl endopeptidase enzyme polynucleotide, each separated by a stretch of contiguous nucleotides which are not complementary to adjacent prolyl endopeptidase enzyme nucleotides, can provide sufficient targeting specificity for prolyl endopeptidase enzyme mRNA. Preferably, each stretch of complementary contiguous nucleotides is at least 4, 5, 6, 7 or 8 or more nucleotides in length. Non-complementary intervening sequences are preferably 1, 2, 3 or 4 nucleotides in length. One skilled in the art can easily use the calculated melting point of an antisense-sense pair to determine the degree of mismatching which will be tolerated between a particular antisense oligonucleotide and a particular prolyl endopeptidase enzyme polynucleotide sequence.

Antisense oligonucleotides can be modified without affecting their ability to hybridize to a prolyl endopeptidase enzyme polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′ substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152158, 1992; Uhlmann et al., Chem. Rev. 90, 543584, 1990; Uhlmann et al., Tetrahedron Lett. 215, 35393542, 1987.

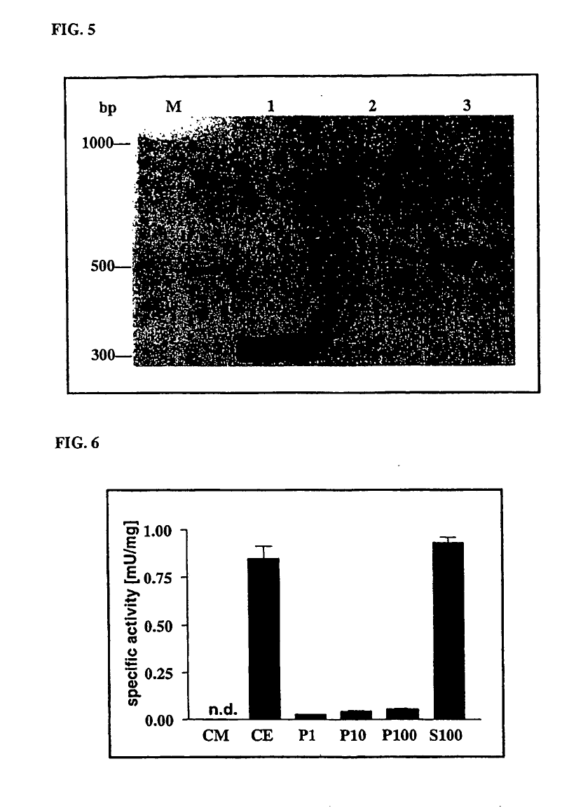

During cultivation of U343 cells, we were not able to detect a significant extracellular activity of PEP, all activity was found in the cytoplasmatic fraction (FIG. 6). Furthermore, overexpression of a PEP-GFP fusion protein revealed an intracellular localization of the enzyme. An unobvious possibility of a relationship may be the intracellular involvement of PEP in the receptor mediated signaling cascade of neuropeptides.

Intriguingly, in the mammalian glioma cell line U343, the IP₃ concentration was found to be increased according to a reduced expression of PEP and dependent on the proteolytic activity being suppressed by an inhibitor (FIG. 3). However, the increased amount of IP₃ observed in the antisense cell lines still leaves the question open as to which domain of PEP is responsible for this effect. The results obtained with the specific inhibitor indicate an involvement of the catalytic domain within the enzyme. The inhibitor employed, Fmoc-Ala-Pyrr-CN, interacts with the enzyme in a substrate like manner and restricts changes to the active site of the enzyme (9;17). This strongly suggests that the impaired proteolytic activity of PEP is responsible for the elevated IP₃ concentration. The release of Ca²⁺ from the endoplasmatic reticulium (ER) is controlled by IP₃ receptors and ryanodine receptors. Hence, an amplification of IP₃ by PEP inhibition may contribute to the intracellular release of Ca²⁺ from the ER.

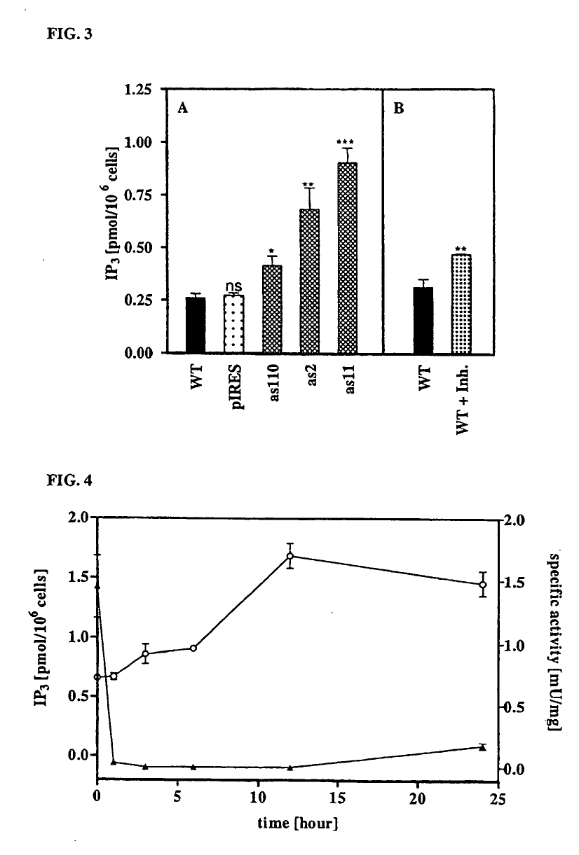

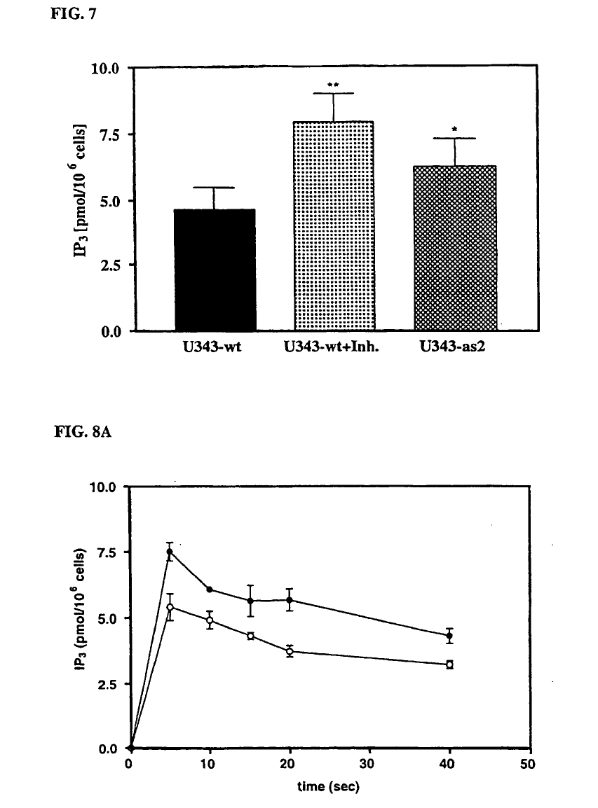

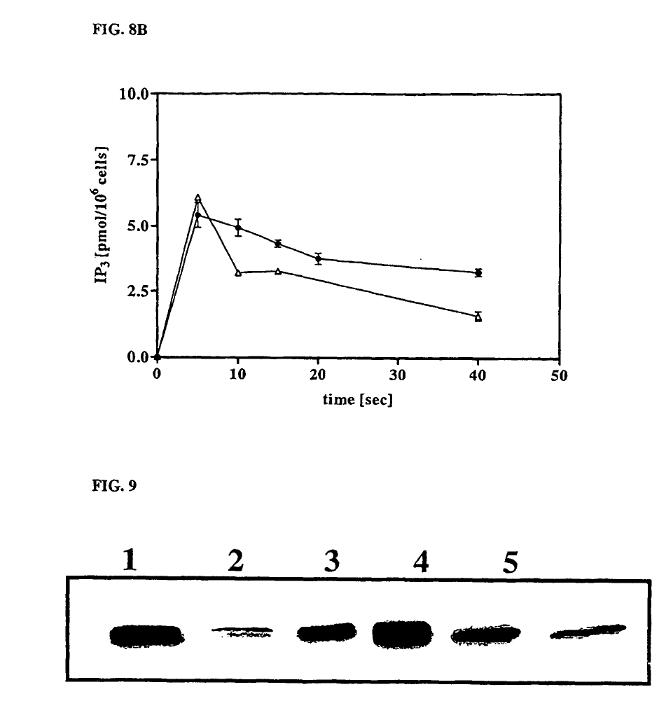

The astro glioma cell line U343 expresses NK-R 1, the specific receptor for the neuropeptide substance P (FIG. 4). Both, U343 antisense cell lines and cells incubated with PEP inhibitor revealed an amplified IP₃ signal after substance P stimulation (FIG. 7), but leaving the kinetic profile of the stimulation unchanged (FIG. 8). This amplification supports that PEP influences the signaling cascade of neuropeptides such as substance P. However, the amplification of the IP₃ signal is partially due to the increased baseline level of the second messenger and partially due to an enhanced efficacy of substance P. Moreover, the observed effect is indirect: The extremely delayed response of IP₃ concentration to total inhibition of PEP confirms that suggestion (FIG. 3). Furtheron the enzymatic activity of PEP can be suppressed by a phosphorylated residue adjacent to the P₁ proline residue.

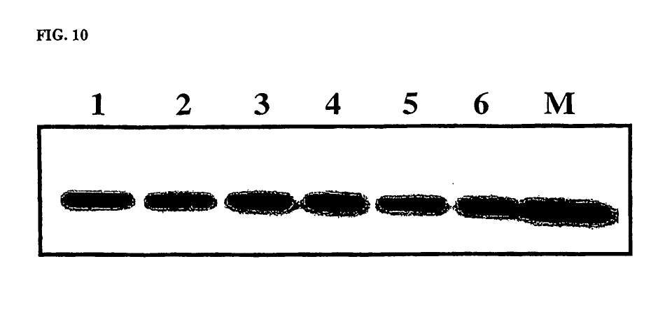

The distribution of PEP is ubiquitous. As shown in Table 2a and 2b in all tested cell lines a reasonable PEP activity could be detected. However, with the exception of the glioma cell line LNZ 308 the brain cell lines display the highest activity. The high distribution of PEP in brain is further confirmed by the high concentration located in different compartments of the rat brain (FIG. 10).

In conclusion, the present results show a novel type of interaction between the signal transduction cascades of neuropeptides such as substance P and the serine peptidase, prolyl endopeptidase. Due to its intracellular localization, the influence of PEP on the signaling cascade offers a new possibility in which way PEP inhibitors can enhance learning and memory. Thus, inhibitors of prolyl endopeptidase can be used for treating impaired learning and/or memory. The inhibition of PEP will, moreover, play a role in other disorders, in which intracellular signaling cascades via modulation of IP₃ concentration are involved. Such disorders are autoimmune diseases, T-lymphocyte mediated immune disorders and tissue regeneration processes, such as wound healing, which are mediated by activated fibroblasts and/or T-lymphocytes. PEP inhibitors may be used in the treatment of these disorders.

In contrast to other proposed methods in the art, the present invention provides an optionally orally available therapy with low molecular weight inhibitors of prolyl endopeptidase. The instant invention represents a novel approach for the treatment of neuronal disorders such as impaired learning and memory, autoimmune diseases and T-lymphocyte mediated immune disorders and tissue regeneration processes, such as wound healing, which are mediated by activated fibroblasts and/or T-lymphocytes.

Known prolyl endopeptidase inhibitors are, e.g. Fmoc-Ala-Pyrr-CN and those listed below:

Z-321
Zeria Pharmaceutical Co Ltd

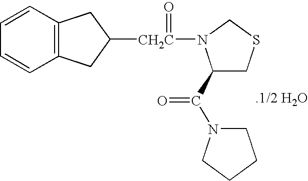

(4R-3-(indan-2-ylacetyl-4-
(1-pyrrolidinyl-carbonyl)-
1,3-thiazolidin
ONO-1603
Ono Pharmaceutical Co Ltd

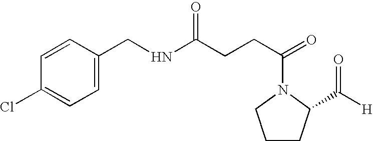

(S)-1-[N-(4-chlorobenzyl)-
succinamoyl]pyrrolidin-2-
carbaldehyd
JTP-4819
Japan Tobacco Inc

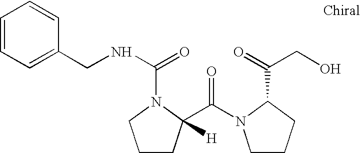

(S)-2-{[(S).(hydroxyacatyl)-1-
pyrrolidinyl]carbonyl}-N-
(phenylmethyl)-1-pyrrolidin-
carboxamid
S-17092
Servier

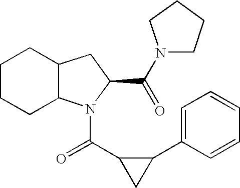

(2S, 3aS, 7aS)-1{[(R,R)-2-
phenylcyclopropyl]
carbonyl}-2-[(thiazolidin-3-
yl)carbonyl]octahydro-1H-
indol

Further prolyl endopeptidase inhibitors are disclosed in JP 01042465, JP 03031298, JP 04208299, WO 0071144, U.S. Pat. No. 5,847,155; JP 09040693, JP 10077300, JP 05331072, JP 05015314, WO 9515310, WO 9300361, EP 0556482, JP 06234693, JP 01068396, EP 0709373, U.S. Pat. No. 5,965,556, U.S. Pat. No. 5,756,763, U.S. Pat. No. 6,121,311, JP 63264454, JP 64000069, JP 63162672, EP 0268190, EP 0277588, EP 0275482, U.S. Pat. No. 4,977,180, U.S. Pat. No. 5,091,406, U.S. Pat. No. 4,983,624, U.S. Pat. No. 5,112,847, U.S. Pat. No. 5,100,904, U.S. Pat. No. 5,254,550, U.S. Pat. No. 5,262,431, U.S. Pat. No. 5,340,832, U.S. Pat. No. 4,956,380, EP 0303434, JP 03056486, JP 01143897, JP 1226880, EP 0280956, U.S. Pat. No. 4,857,537, EP 0461677, EP 0345428, 4JP 02275858, U.S. Pat. No. 5,506,256, JP 06192298, EP 0618193, JP 03255080, EP 0468469, U.S. Pat. No. 5,118,811, JP 05025125, WO 9313065, JP 05201970, WO 9412474, EP 0670309, EP 0451547, JP 06339390, U.S. Pat. No. 5,073,549, U.S. Pat. No. 4,999,349, EP 0268281, U.S. Pat. No. 4,743,616, EP 0232849, EP 0224272, JP 62114978, JP 62114957, U.S. Pat. No. 4,757,083, U.S. Pat. No. 4,810,721, U.S. Pat. No. 5,198,458, U.S. Pat. No. 4,826,870, EP 0201742, EP 0201741, U.S. Pat. No. 4,873,342, EP 0172458, JP 61037764, EP 0201743, U.S. Pat. No. 4,772,587, EP 0372484, U.S. Pat. No. 5,028,604, WO 9118877, JP 04009367, JP 04235162, U.S. Pat. No. 5,407,950, WO 9501352, JP 01250370, JP 02207070, U.S. Pat. No. 5,221,752, EP 0468339, JP 04211648 and WO 9946272, the teachings of which are herein incorporated by reference in their entirety, especially concerning these inhibitors, their definition, uses and their production.

According to a further embodiment the present invention refers to the use of a combination of

a) a peptide hormone, and/or

b) a prolyl endopeptidase inhibitor.

Thus, any combination of a) and any prolyl endopeptidase inhibitor of b) is possible according to the invention.

The daily dosage of the peptide hormone like substance P may be varied over a wide range from 1 nM to 10 μM per kg bodyweight per day, preferably from 100 nM to 1 μM per kg per day, more preferably from 300 nM to 500 nM per kg per day.

Peptide hormones are, e.g. Angiotensin I, Bradykinin potentiating peptide (BPP), Bradykinin, Luliberin, Melanotropin, Neurotensin, Oxytocin, Substance P, Thyroliberin, Tuftsin, or Vasopressin.

The combined use of at least two of the above compounds has the advantage that the duration of action of the active ingredients, the onset of action and the site-specificity can be regulated in a selective manner, which is optimal for each patient or disease. Moreover, the combined administration of substance P and at least one prolyl endopeptidase inhibitor unexpectedly leads to a synergistic effect of an increased concentration of IP₃ compared to the use of either substance P or a prolyl endopeptidase inhibitor alone. More specifically, the balance of the intracellular concentrations of inositol polyphosphates, such as IP₃, IP₄, IP₅, IP₆, etc. is changed to an IP₃ surplus due to the administration of substance P and/or a prolyl endopeptidase inhibitor. Higher inositol polyphosphates, such as IP₄, IP₅, IP₆, etc. are regulators of inositol phosphate binding proteins. Such inositol polyphosphate binding proteins are for instance synaptotagmine, the GTPase-activating proteins Gap1^{IP4BP} and Gap1^{m}, Bruton's tyrosine kinase (BtK), proteolipid-protein (PLP), vinculin, centaurin α, Golgi coatomer, p130, AP-2 and AP-3. We have found that the above mentioned inositol polyphosphate binding proteins are involved in intracellular vesicle transport processes, especially in the release of neurotransmitters, in the organization of the cyto-skeleton and are, therefore, involved in neurodegenerative diseases, diseases of specific tissues and cell cycle regulation.

Thus, neuronal disorders such as impaired learning and memory, autoimmune diseases and T-lymphocyte mediated immune disorders and tissue regeneration processes, such as wound healing, which are mediated by activated fibroblasts and/or T-lymphocytes, can be treated in a very specific manner: When substance P is used in combination with a PEP-inhibitor the amelioration occurs immediately.

Moreover, direct onset of the treatment/improvement in combination with a long term therapy of neuronal disorders, such as impaired learning and memory, autoimmune diseases and T-lymphocyte mediated immune disorders and tissue regeneration, such as wound healing can be obtained by using an inhibitor of PEP in combination with a substance P containing medicament.

The utility of the compounds useful as PEP inhibitors to modulate intracellular IP₃ concentration and, subsequently, intracellular signaling cascades, which are involved in several disorders, like neuronal disorders such as impaired learning and memory, autoimmune diseases and T-lymphocyte mediated immune disorders and tissue regeneration processes, such as wound healing, which are mediated by activated fibroblasts and/or T-lymphocytes, can be determined according to the procedures described in Examples 2 and 3. The present invention therefore provides a method of treating a condition mediated by modulation of the PEP activity in a subject in need thereof which comprises administering any of the compounds or pharmaceutical compositions as defined herein in a quantity and pharmaceutically acceptable composition effective to treat the condition. Additionally, the present invention includes the use of such a compound for the preparation of a medicament for the treatment of a condition mediated by modulation of the PEP activity in a subject. The compound may be administered to a patient by any conventional route of administration, including, but not limited to, intravenous, oral, subcutaneous, intramuscular, intradermal and parenteral.

Preferred PEP inhibitors are substituted aminoketones, e.g. Benzyl-N-[1-(cyclopentylcarbonyl)-2-methylbutyl]carbamate.

The present invention further provides screening methods for test compounds which bind to or modulate the activity or expression of prolyl endopeptidase. A test compound preferably binds to prolyl endopeptidase or to the prolyl endopeptidase coding gene. More preferably, a test compound decreases prolyl endopeptidase activity by at least about 10, preferably about 50, more preferably about 75, 90 or 100% relative to the absence of the test compound. Most preferably, a test compound decreases prolyl endopeptidase activity by at least about 10, preferably about 50, more preferably about 75, 90 or 100% and increases the concentration of intracellular inositol (1,4,5) triphosphate concentration by at least about 1fold, preferably about 2fold, more preferably about 3fold, 4fold or higher relative to the absence of the test compound.

The screening methods combine the following steps:

cells containing inositol (1, 4, 5) triphosphate and prolyl endopeptidase are provided, e.g. selected from, but not restricted to, the human glioma cell line U-343, the human neuroblastoma cell line SH-SY5Y and the human astroglioma cell line LN-405,

the inositol (1,4,5) triphosphate concentration and the prolyl endopetidase activity of the cells are measured. Basal levels for substance P concentration and prolyl endopeptidase activity are 0.3 pmol/10⁶ cells and 20-40 mU/mg cell extract respectively,

the cells are incubated with a test compound,

the inositol (1,4,5) triphosphate concentration is measured,

optionally the residual prolyl endopeptidase activity is measured, and

optionally a prolyl endopeptidase inhibitor is isolated.

Optionally, the efficiency of test compounds regarding their IP₃ concentration raising potential can be screened in combination with the administration of a peptide hormone like substance P:

cells containing inositol (1, 4, 5) triphosphate and prolyl endopeptidase are provided, e.g. selected from, but not restricted to, the human glioma cell line U-343, the human neuroblastoma cell line SH-SY5Y and the human astroglioma cell line LN-405,

the inositol (1,4,5) triphosphate concentration and the prolyl endopetidase activity of the cells are measured. Basal levels for the peptide hormone (e.g. substance P) concentration and prolyl endopeptidase activity are 0.3 pmol/10^{6 cells} and 20-40 mU/mg cell extract respectively,

the cells are incubated with a test compound in combination with the peptide hormone like substance P,

the inositol (1, 4, 5) triphosphate concentration is measured,

optionally the residual prolyl endopeptidase activity is measured, and

optionally a prolyl endopeptidase inhibitor is isolated.

Preferably, prolyl endopeptidase inhibitors are isolated which provide for a higher inositol (1,4,5) triphosphate concentration than known prolyl endopeptidase inhibitors.

Test compounds can be pharmacological agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead-one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des., 12, 145, 1997.

The present invention also provides pharmaceutical compositions comprising one or more compounds of this invention especially a PEP inhibitor and/or a peptide hormone like substance P in association with a pharmaceutically active carrier.

To prepare the pharmaceutical compositions of this invention, one or more active compounds or salts thereof of the invention as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included.

Injectable suspensions may also prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.03 mg to 100 mg/kg (preferred 0.1-30 mg/kg) and may be given at a dosage of from about 0.1-300 mg/kg/day (preferred 1-50 mg/kg/day). The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using conventional methods known from the art.

The method of treating conditions modulated by the prolyl endopeptidase described in the present invention may also be carried out using a pharmaceutical composition comprising any or any combination of the compounds as defined herein and a pharmaceutically acceptable carrier. The pharmaceutical composition may contain between about 0.01 mg and 100 mg, preferably about 5 to 50 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds, mixtures or compositions of the present invention may be administered in a single daily dose, or the total daily dosage may be administered in divided doses of two, three or four times daily. Furthermore, compounds, mixtures or compositions for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen.

For instance, for oral administration in the form of a tablet or capsule, the active drug component, mixtures or compositions can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound, mixture or composition of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Compounds, mixtures or compositions of the present invention may also be delivered by the use of monoclonal antibodies as individual carriers to which the compound molecules are coupled. The compounds, mixtures or compositions of the present invention may also be coupled with soluble polymers as targetable drug carriers. Such polymers can include polyvinylpyrrolidone, pyran copolymer, polyhydroxypropylmethacrylamidephenol, polyhydroxyethylaspartamid-ephenol, or polyethyl eneoxidepolyllysine substituted with palmitoyl residue. Furthermore, the compounds, mixtures or compositions of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds, mixtures or compositions of this invention may be administered in any of the foregoing compositions and according to dosage regimens established in the art whenever treatment of the addressed disorders is required.

The daily dosage of the products may be varied over a wide range from 0.01 to 1.000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of disease condition. In addition, factors associated with the particular patient being treated, including patient age, weight, diet and time of administration, will result in the need to adjust dosages.

In general, to prepare the pharmaceutical compositions of this invention, one or more active compounds or salts thereof of the invention as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.03 mg to 100 mg/kg (preferred 0.1-30 mg/kg) and may be given at a dosage of from about 0.1-300 mg/kg/day (preferred 1-50 mg/kg/day). The dosages, however, may be varied depending upon the requirement of the patients, the severity of the condition being treated and the compound being employed. The use of either daily administration or post-periodic dosing may be employed.

### EXAMPLES OF THE INVENTION

### Example 1

Suppression of Prolyl Endopeptidase Expression in U343 Cells

A cell line displaying sufficiently high concentrations of PEP was neccessary to investigate the cellular role of prolyl endopeptidase. The astro glioma cell line U343 displayed the highest amount of active PEP out of six cell lines (CaCo, EFE, SNB19, U343, U937, SY5Y) investigated by measuring the hydrolytic activity of the cell lysate on the fluorogenic substrate Z-Gly-Pro-NHMec. Additionally, Western-blot analysis confirmed the high amount of PEP expression. U343 cells express prolyl endopeptidase in a concentration of approximately 5 mU/mg total protein, enabling the detection of changes in concentration and activity by our methods.

Two different approaches were chosen to influence the intracellular activity of PEP in U343 cells. Fmoc-Ala-Pyrr-CN is a potent and specific inhibitor for PEP (9), with an observed Kᵢ value of 70 pM against recombinant human PEP (data not shown). It has been shown that this inhibitor is able to penetrate the cell membrane and inhibit PEP intracellularly (10). Incubating U343 cells in Fmoc-Ala-Pyrr-CN containing medium (5 μM), total inhibition of intracellular PEP activity is achieved within 5 min. This inhibition is observed for up to 12 hours without adding fresh inhibitor. In addition, a totally different approach of reducing PEP activity was employed by generating antisense cell lines displaying reduced expression of the target enzyme.

To obtain the coding sequence for the catalytic domain of PEP, total RNA of 1×10⁷ cells from the human glioma cell line U343 was isolated with TRIzol® Reagent (GIBCO BRL). Four μg of the obtained total RNA was converted into cDNA by RT-PCR using hexanucleotide primers and M-MLV reverse transcriptase (Promega). The resulting cDNA pool (4 μl) was then amplified with the Expand™ PCR System (Roche) using a pair of PEP specific primers (5′-CATATGCTGTCCTTCCAGTACC-3′; 5′-GATTCCGCTGTCAGGAGGAAGCACG-3′). The resulting PCR fragment contained the entire open reading frame. By PCR using two nested primers (5′-CATATGGGAATTGATGCTTCTGATTAC-3′; 5′-GAATTCTGGAATCCAGTC GACATTCAG-3′) a 0.9 kb fragment was generated containing the catalytic domain of the enzyme (amino acids 442-731 of human PEP). This fragment was cloned into pPCR-Script Cam (Stratagene). The EcoRI restriction sites of the subcloned vector and of the nested reverse primer were used to ligate the fragment into the mammalian expression vector pIRESneo (Clontech). The resulting transformants were analyzed by PCR to determine if the insert was present in antisense orientation and the correct nucleotide sequence was verified by DNA sequencing (GATC Biotech AG).

For Western-blot analysis, a polyclonal antibody against human PEP was generated. Therefore, rabbits were immunized with a peptide containing the N-terminal PEP sequence of amino acids 10-25. Specific antibodies (S449) were purified from rabbit serum using an affinity chromatography column with the immobilized peptide. Analytical electrophoresis in SDS-polyacrylamide gels was performed as described by Laemmli with separation gels containing 12% acrylamide (7). The separated cell extracts were transferred to a nitrocellulose membrane (Schleicher&Schuell) following standard procedure (8). PEP and actin were detected by the polyclonal antibody S449 (1:400) and monoclonal antibody ANTI-ACTIN (1:2500, Sigma, A2066), respectively, and visualized by chemiluminescence according to manufacturers protocol (SuperSignal™ West Pico, PIERCE). Semi quantitative analysis of Western-blot results was done by densitometry software (Gelscan 3D, BioSciTec).

After verifying the correct sequence, U343 cells were transfected with this vector. Therefore, the human glioma cell line U343 was maintained in DMEM medium containing 10% fetal bovine serum (GIBCO BRL) and 60 μg/ml gentamycin (GIBCO BRL) at 37° C. in a 5% CO₂ atmosphere. The mammalian expression vectors were transfected into U343 cells using Polyfectin-reagent (BIONTEX) according to the manufacturer's protocol. Stable transfectants were selected in media containing 400 μg/ml G418 (Duchefa). Taking advantage of the neomycin resistance for positive transformants, 120 clones were isolated using cloning rings. Out of these clones, 8 stable cell lines were established and all of them revealed a reduced PEP activity (Table 1). However, antisense cell lines 1, 13 and 110 lost their antisense effect during the extended time of cultivation. Most of the established cell lines displayed a reduced PEP activity about 50%.

For the determination of PEP activity, U343 wild-type and PEP antisense cells (1×10⁷) were harvested by washing twice in phosphate buffered saline (GIBCO BRL) and resuspended in 200 μl assay buffer (50 mM HEPES pH 7.5; 200 mM NaCl; 1 mM EDTA; 1 mM DTT). Cell lysis was achieved by three cycles of thawing and freezing and then the cells were removed from the incubation flask by a cell scraper. The obtained lysate was centrifuged at 13000 rpm for 1 min and the supernatant transferred into a fresh tube. All steps were performed on ice. The protein concentration in the supernatant was determined according to the method of Bradford (5). PEP activity was measured in assay buffer using the fluorogenic substrate Z-Gly-Pro-NHMec (10 μM) (Bachem) on a Kontron spectrofluorometer SFM 25 (excitation 380, emission 460) equipped with a four-cell changer and controlled by an IBM-compatible personal computer. The obtained data were analyzed with the software Flucol (6).

Cell line as-11 showed the strongest reduction in PEP activity, 30% remaining activity compared to wild-type U343 cells. Using Western-blot analysis, the results obtained by activity measurements could be confirmed (FIG. 2, Table 1). In all antisense cell lines, the reduced proteolytic activity was due to the decreased expression of PEP. As a control, cells transformed with the insert free vector pIRES revealed unchanged PEP activity and expression (FIG. 2). The generated antisense cell lines did not display a common change in phenotype, however individual changes were observed (Table 1). U343 as-11 cell line displayed an increased trypsin sensitivity, an increased volume of the cells (3 fold), and was no longer able to grow to 100% confluence.

TABLE 1
PEP remaining activities and expression patterns in human
glioma U343 anti-sense cell lines
All anti-sense cell lines show a reduced remaining activity
and expression intensity in comparison to wild-type cell
line U343; remaining activity = percentage of activity
regarding to wild-type U343 cells; remaining expression =
densitometric analysis of Western-blot, n = 2.

| | | remaining | remaining |
|---|---|---|---|
| | specific activity | activity | expression |
| cell line | (mU/mg) | (%) | (%) |
| U343-wt | 5.00 ± 0.14 | 100.0 | 100.0 |
| U343-as2 | 2.64 ± 0.08 | 53.0 | 57.0 |
| U343-as11* | 1.52 ± 0.04 | 30.0 | 20.0 |
| U343-as40 | 2.70 ± 0.06 | 54.0 | 65.0 |
| U343-as60 | 2.12 ± 0.04 | 42.0 | 33.0 |
| U343-as70 | 2.18 ± 0.08 | 43.0 | 61.0 |
| U343-as110 | 4.20 ± 0.10 | 84.0 | 71.0 |

*changes in phenotype

### Example 2

Modulation of IP₃ Concentration Dependent on Prolyl Endopeptidase

To characterize the intracellular function of PEP, the IP₃ concentration in the antisense cell lines Was measured. Cells were grown in 25 cm² culture flasks to nearly 100% confluence. IP₃ concentration was determined by an isotope dilution method (Amersham Phamacia Biotech) using 0.5×10⁶ cells per measurement. To inhibit intracellular PEP, the cells were washed twice with PBS and incubated for 4 hours in Optimem 1 medium (GIBCO BRL) supplemented with 5 μM PEP-inhibitor Fmoc-alanyl-pyrroline-2-nitrile (Fmoc-Ala-Pyrr-CN). All measurements were done in quadruplicate. The calculation of IP₃ concentration and the statistical analysis (t-test) were performed using Prism 3.0 (Graph Pad Software).

The IP₃ concentration detected in U343 wild-type cells was approximately 0.26±0.02 pmol/10⁶ cells (n=4). The cell line transfected by the insert free vector pIRES revealed no significant changes in IP₃ concentration. However, the second messenger concentration was found to be increased in all generated antisense cell lines (FIG. 3a). A slight, but significant elevation of approximately 0.15±0.04 pmol/10⁶ cells (n=4) could be observed with cell line as-110 displaying only mildly reduced PEP activity. A stronger increase in IP₃ concentration could be observed in antisense cell lines having remaining PEP activity of 50% and lower (FIG. 2). U343 cell line as-2 (53% remaining activity, 57% remaining expression) revealed an increase of up to 2.5 fold in IP₃ concentration. In cell line as-11 (30% remaining activity, 20% remaining expression) the IP₃ concentration was found to be even higher, about 3.5 fold. Together, this data show a correlation between a decreasing PEP concentration and an increased IP₃ concentration.

To use an alternative approach suppressing PEP activity in U343 cells, the cells were incubated for 3 hours in presence of the specific inhibitor Fmoc-Ala-Pyrr-CN (5 μM). The influence of DMSO, necessary for dissolving the inhibitor, was excluded by adding the same amount (0.05%) of DMSO to the media of control cells. In confirmation of the results obtained with the antisense cell lines, the basal IP₃ concentration was found to be increased in PEP inhibitor treated cells (FIG. 4). However, the observed changes in IP₃ concentration were only 0.16 pmol/10⁶ cells. This is much lower than the observed change on IP₃ concentration in cell line as-11 still having 30% remaining PEP activity, and calls into question the correlation between PEP activity and IP₃ concentration. Therefore, the amount of the second messenger was investigated over an extended time period of total inhibition. As shown in FIG. 4, the IP₃ concentration increased during the time of incubation. The maximum concentration reached after 12 hours total inhibition coincides with the period of total inhibition achieved without adding fresh inhibitor. Moreover, the lower concentration of IP₃ measured after 24 hours reflects the slight recovery of PEP activity at that time point. The total amount of IP₃ after 12 hours (1.69±0.1) pmol/10⁶ cells; n=4) is higher than the concentration measured in cell line as-11 (0.9±0.07 pmol/10⁶ cells; n=4), displaying 30% remaining enzyme activity.

### Example 3

Prolyl Endopeptidase Dependent IP₃-Accumulation After Stimulation by Substance P

To investigate whether the observed effect on IP₃ concentration may represent a novel interaction between the biological activity of neuropetides and PEP, substance P was chosen to stimulate U343 cells.

Wild-type and PEP antisense U343 cell lines were cultured in duplicate in 21 cm² culture dishes (Greiner) until confluence. Prior to stimulation the cells were washed twice in PBS and preincubated for 10 h in Optimem1 medium containing 1.6 μg/ml leupeptin (Sigma), 0.86 μg/ml chymostatin (Sigma), and 40 μg/ml bacitracin (Sigma) at 37° C. and 5% CO₂. Substance P (Bachem) was added to obtain a final concentration of 1×10⁻⁶ M and the incubation was stopped at the indicated time by rapidly aspirating the medium and adding of 0.4 ml ice cold trichloric acid. Preparation of samples and measurement of IP₃ concentration were performed as described above.

Using RT-PCR, the occurrence of substance P specific neurokinin receptors (NK-R) in U343 cells was investigated. PCR products could be obtained for two receptors displaying an appropriate size (FIG. 5). Sequencing analysis of these PCR products revealed that the mRNA's of NK-R 1 and NK-R 3 are present in U343 cells. Being aware that substance P is an excellent in vitro substrate for PEP, we investigated potential degradation of substance P during the incubation time in the serum free Optimem1 medium of U343 cells by MALDI-TOF MS analysis. However, during the used incubation time of 10 min, no PEP specific degradation was observed (data not shown). This is in agreement with the fact that no PEP activity is measurable in the medium (FIG. 6).

Stimulation of the U343 cells with 1 μM substance P for 5 sec led to a rise in IP₃ concentration. In the case of wild-type U343 cells, substance P stimulation raised the IP₃ concentration about 14 fold, to a value of 5.4±0.49 pmol/10⁶ cells (n=4) (FIG. 7). In parallel, U343 cells treated with Fmoc-Ala-Pyrr-CN and cell line as-2 revealed a higher concentration of IP₃ after substance P stimulation. Comparing the total values after substance P stimulation, the IP₃ concentration again correlated with the impaired PEP activity (FIG. 7) and the concentration difference is even greater. The difference was 1.7 pmol/10⁶ cells to 7.9 pmol/10⁶ cells in non stimulated and stimulated cells, respectively. However estimating the IP₃ concentration proportionally, the observed difference by inhibition decreased from 5.5 fold to 1.7 fold in non stimulated to stimulated cells, respectively.

In FIG. 8, the change of IP₃ concentration over the time of substance P stimulation is illustrated. To compare the stimulation dependent increase of IP₃ concentration, the amount of IP₃ in the non stimulated state was subtracted as a baseline. All three cell lines, U343 wild-type untreated or inhibitor treated and as-2 cells, displayed a similar stimulation pattern over the time (FIG. 8a, b). The measured maximum in IP₃ concentration always occurred after 5 sec stimulation. The stimulation displays a rapid increase of the second messenger concentration followed by a slow decline, not reaching baseline levels after 40 sec. Whereas the difference between U343 wild-type and as-2 revealed no consistent difference in IP₃ concentration (FIG. 8b); the inhibitor treated cells displayed during the whole time of incubation an increased stimulation of IP₃ by substance P (FIG. 8a).

### Example 4

Distribution of Prolyl Endopeptidase in Different Mammalien Cell Lines

In order to find out the distribution of prolyl endopeptidase activity in several human tissues, different cell culture cryo-stocks and cells, grown in monolayers, were examined.

For homogenization, U343 wild-type and PEP antisense cells (1×10⁷) were harvested by washing twice in phosphate buffered saline (GIBCO BRL) and resuspended in 200 μl assay buffer (5 mM HEPES pH 7.5; 200 mM NaCl; 1 mM EDTA; 1 mM DTT). The frozen cell culture stocks were thaw rapidly at 37° C. and centrifuged at 500 g for 10 min. The cell pellets were resuspended in 100 μl assay buffer. Cell lysis was achieved by three cycles of thawing and freezing and then the cells were removed from the incubation flask by a cell scraper. The obtained lysate was centrifuged at 13000 rpm for 1 min and the supernatant transferred into a fresh tube. All steps were performed on ice. The protein concentration in the supernatant was determined according to the method of Bradford (5). PEP activity was measured in assay buffer using the fluorogenic substrate Z-Gly-Pro-NHMec (10 μM) (Bachem) on a Kontron spectrofluorometer SFM 25 (excitation 380, emission 460) equipped with a four-cell changer and controlled by an IBM-compatible personal computer. The obtained data were analyzed with the software Flucol (6).

Western-blot analysis (see FIG. 9) was performed according to the methods described in Example 1. The PEP-signals, detected in 6 cultivated human cell lines (1 U343, 2 LN-405; 3 SH-SY5Y; 4 BeWo; 5 U-138-MG; 6 CACO-2) correlates with the PEP-activity, measured in these. No signals at all could be detected in non-cultivated cryo-stocks. Cytosolic supernatants from each cell lines were extracted and analyzed as described in Example 1. Ten μg (lane 1-3), 20 μg (lane 5) and 40 μg (lane 4, 6) of cytosolic supernatants were loaded per lane. Western-blot was incubated with PEP specific antibody S449 (1:400) and detected by chemiluminescence technique.

TABLE 2A
PEP-activities in cultivated human cell lines
All cell lines show PEP-activity. The percentage of
PEP-activities relates to the human glioma cell line
U343, which dsiplays the highest amount of PEP-activity.
Data are obtained in duplicate (mean ± SD).

| | | specific activity | activity |
|---|---|---|---|
| cell line | cell type | (mU/mg) | (%) |
| U-343 | glioma | 2.30 ± 0.082 | 100.0 |
| LN-405 | astrocytoma | 1.65 ± 0.037 | 72.0 |
| SH-SY5Y | neuroblastoma | 1.39 ± 0.029 | 60.0 |
| U-138-MG | glioblastoma | 0.74 ± 0.0525 | 32.0 |
| BeWo | choriocarcinoma | 1.10 ± 0.070 | 48.0 |
| CACO-2 | colon | 0.57 ± 0.081 | 25.0 |
| | adenocarcinoma | | |

TABLE 2B
PEP-activities in non-cultivated human cryo-stocks
ALL CRYO-STOCKS SHOW PEP-ACTIVITY. THE
PERCENTAGE OF PEP-ACTIVTIES IS SHOWN IN
RELATION TO THE HUMAN GLIOMA CELL LINE
U343, WHICH DISLAYS THE HIGHEST AMOUNT OF
PEP-ACTIVITY. DATA ARE OBTAINED IN DUPLICATE
(MEAN ± SD).

| | | specific activity | activity |
|---|---|---|---|
| cell line | cell type | (mU/mg) | (%) |
| MDA-MB-435S | breast carcinoma | 1.96 ± 0.000 | 85.0 |
| COLO-699 | lung adenocarcinoma | 0.84 ± 0.007 | 36.0 |
| ECV-304 | umbilical cord | 0.79 ± 0.069 | 34.0 |
| | endothelial cells | | |
| A-431 | epidermoid carcinoma | 0.60 ± 0.027 | 26.0 |
| A-549 | lung carcinoma | 0.58 ± 0.021 | 25.0 |
| HEK 293 | embryonal kidney | 0.55 ± 0.003 | 24.0 |
| T-Zellen, human, | primary culture | 0.42 ± 0.000 | 18.0 |
| female | | | |
| DV 145 | prostate carcinoma | 0.41 ± 0.009 | 18.0 |
| COLO-677 | small cell lung | 0.28 ± 0.007 | 12.0 |
| | carcinoma | | |
| CAKI-1 | kidney carcinoma | 0.24 ± 0.001 | 10.0 |
| CAKI-2 | kidney carcinoma | 0.23 ± 0.005 | 10.0 |
| PC 3 | prostate | 0.17 ± 0.007 | 7.5 |
| | adenocarcinoma | | |
| LNZ 308 | glioma | 0.07 ± 0.003 | 3.0 |
| RCC 100 | renal cell carcinom | 0.04 ± 0.008 | 2.0 |

### Example 5

Distribution of Prolyl Endopeptidase in Brain of Male Wistar Rats

In order to find out the distribution of prolyl endopeptidase activity in several human tissues, different cell culture cryo-stocks and cells, grown in monolayers, were examined.

The frozen rat tissue samples were homogenize in a micro-mortar (Roth) and resuspended in 400 μl assay buffer (50 mM HEPES pH 7.5; 200 mM NaCl; 1 mM EDTA; 1 mM DTT).

The suspension were centrifuged at 13000 rpm for 3 min and the supernatant transferred into a fresh tube. For further procedure see under “homogenization of cells grown in monolayer” in Example 4.

After Western-blot analysis (see FIG. 10) of the PEP-expression in rat brain, the PEP-concentration, measured in each brain-regions correlates with the signals in the Western-blot analysis. Tissue from each brain-regions (1 cortex, 2 hippocampus, 3 medulla oblongata, 4 cerebellum, 5 thalamus, frontal lobe) were extracted and analyzed as described in Example 1. Thirty μg of total protein were loaded per lane. Cytosolic U343 cell line supernatant was used as positive control (M, 20 μg/lane). Western-blot was incubated with PEP specific antibody S449 (1:400) and detected by chemiluminescence technique.

### Example 6

Synthesis of Benzyl-N-[1-(cyclopentylcarbonyl)-2-methylbutyl]carbamate Z-isoleucinal 2

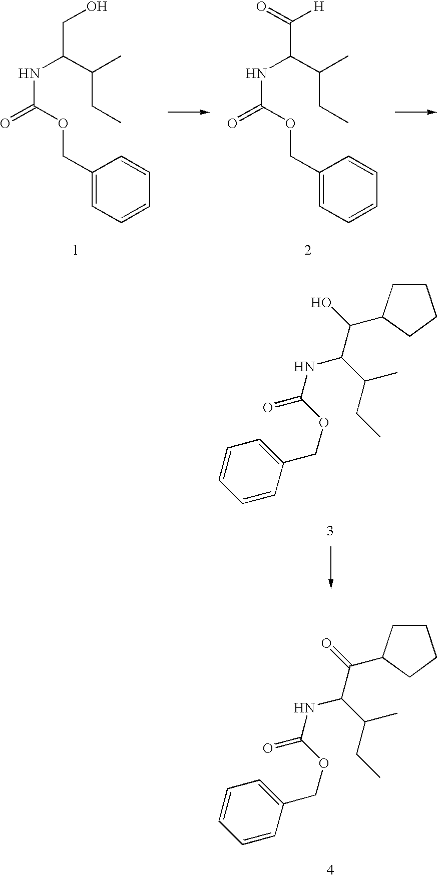

Oxalylchloride (714 μl, 8.28 mmol) was dissolved 10 ml of dry dichlormethane and brought to −78° C. Then DMSO (817 μl, 8.28 mmol) was added dropwise. The solution was stirred for 20 min at −78° C. Then 1 (1.22 g, 4.6 mmol) was added and the mixture was stirred for 20 min. After that TEA (2.58 ml, 18.4 mmol) was added and the mixture was allowed to reach r.t. The mixture was diluted with hexane/ethylacetate (2/1 v/v) and 10 ml of HCl (10% in water) was added. The organic layer was separated and the aqueous phase was extracted with 20 ml of methylenechloride. All organic layers where collected and whashed with brine, followed by water, then dried.

The product was purified by column chromatography using silica gel and heptane/chloroform.

Yield: 0.59 g, 52%

Benzyl-N-1-[cyclopentyl(hydroxy)methyl]-2-methylbutylcarbamate 3

2 (0.59 g, 2.42 mmol) was dissolved in 10 ml of dry THF and cooled down to 0° C. Then cyclopentylmagnesiumbromide (1.45 ml of a 2M solution) where added. After completion of the reaction water (2 ml) was added and solution was neutralized by adding aqueous HCl. Then methylenechloride was added and the organic layer was separated and dried (Na₂SO₄). After evaporation the resulting oil was used without further characterisation.

Benzyl-N-[1-(cyclopentylcarbonyl)-2-methylbutyl]carbamate 4

3 (0.683 g, 2.15 mmol) was treated like 1. Oxalylchloride (333 μl, 3.87 mmol), DMSO (382 μl, 5.37 mmol), TEA (1.2 ml, 8.59 mmol)

Yield: 0.203 g, 30%

### Example 7

Determination of IC₅₀-Value for Benzyl-N-[1-(cyclopentylcarbonyl)-2-methylbutyl]carbamate

100 μl inhibitor stock solution were mixed with 100 μl buffer (EPES pH7.6) and 50 μl substrate (Z-Gly-Pro-AMC, final concentration 10 μM). Reaction was started by addition of 20 μl purified recombinant human prolyl endopeptidase. Fluorescence measurement was performed at 37° C. and λₑₓᵢₐₜₐₜᵢₒₙ=380 nm, λₑₘᵢₛₛᵢₒₙ=465 nm for 60 min using a plate reader (HTS7000plus, Applied Biosystems, Weiterstadt, Germany). The final inhibitor concentrations ranged between 1 pM and 10 μM. For calculation of the IC₅₀ value Prism 3″ (Graphpad) was used. The IC₅₀-value of Benzyl-N-[1-(cyclopentylcarbonyl)-2-methylbutyl]carbamate was calculated as of IC₅₀=8,91*10⁻⁷ M

Footnotes

The abbreviations used are:

AVP Arginine-vasopressin

ER endoplasmic reticulum

Fmoc 9-fluorenylmethyloxycarbonyl

Fmoc-Ala-Pyrr-CN Fmoc-alanyl-pyrroline-2-nitrile

IP₃ Inositol (1,4,5) triphosphate

LTP Long-term potentiation

MALDI-TOF Matrix-assisted laser desorption/ionization-time of flight

MIPP Multiple inositol polyphosphatase

MS Mass spectrometry

NHMec 7-(4-methyl) coumaryl-amide

PEP Prolyl endopeptidase

PLC Phospholipase C

Z Benzyloxycarbonyl

1. A method of using an inhibitor of prolyl endopeptidase for the modulation of the intracellular level of inositol (1,4,5) triphosphate.

2. The method according to claim 1, wherein the inhibitor of prolyl endopeptidase is used in combination with peptide hormones.

3. The method according to claim 1, wherein said prolyl endopeptidase is present within cells and said inhibitor of prolyl endopeptidase penetrates into the cells to act on prolyl endopeptidase enzymatic activity whereby the intracellular level of inositol (1,4,5) triphosphate is modulated.

4. The method according to claim 1 wherein said inhibitor is used for preventing or treating a condition selected from the group consisting of impaired learning and memory, autoimmune diseases and T-lymphocyte mediated immune disorders and tissue regeneration processes mediated by activated fibroblasts and/or T-lymphocytes.

5. The method according to claim 1, wherein said inhibitor of proline endopeptidase is in combination with a physiologically compatible drug delivery vehicle.

6. The method according to claim 1, wherein said inhibitor of proline endopeptidase is brought into contact with inositol (1,4,5) triphosphate through a route selected from group consisting of parenteral, enteral, oral, inhalation and suppository.

7. The method according to claim 1, wherein the inhibitor of prolyl endopeptidase is used in combination with substance P.

8. The method according to claim 1, wherein the prolyl endopeptidase inhibitor is Fmoc-Ala-Pyrr-CN.

9. The use method according to claim 1, wherein the prolyl endopeptidase inhibitor is selected from the group consisting of (4R)-3-(indan-2-ylacetyl)-4-(1 pyrrolidinyl-carbonyl)-1,3-thiazolidin (Z-321, ZeriaPharmaceutical Co Ltd.); (S)-1-[N-(4-chlorobenzyl)-succinamoyl]pyrrolidin-2-carbaldehyd (ONO-1603, Ono Pharmaceutical Co Ltd.); (S)-2-{[(S).(hydroxyacatyl)-1-pyrrolidinyl]carbonyl}-N-(phenylmethyl)-1-pyrrolidin-carboxamid (JTB-4819, Japan Tobacco Inc.) and (2S,3aS,7aS)-1 {[(R,R)-2-phenylcyclopropyl]carbonyl}-2-[(thiazolidin-3-yl)carbonyl]octahydro-1H-indol (S-17092, Servier).

10. A method of screening for prolyl endopeptidase inhibitors, comprising the steps of
providing cells containing inositol (1,4,5) triphosphate and prolyl endopeptidase;
measuring the inositol (1,4,5) triphosphate concentration and the prolyl endopeptidase activity of the cells;
incubating the cells with a test compound;
measuring the inositol (1,4,5) triphosphate concentration; and
isolating a prolyl endopeptidase inhibitor.

11. A screening method for prolyl endopeptidase inhibitors, comprising the steps of
providing cells containing inositol (1,4,5) triphosphate and prolyl endopeptidase;
measuring the inositol (1,4,5) triphosphate concentration and the prolyl endopeptidase activity of the cells;
incubating the cells with a test compound in combination with substance P;
measuring the inositol (1,4,5) triphosphate concentration; and
isolating a prolyl endopeptidase inhibitor.

12. The screening method according to claims 10 or 11, wherein prolyl endopeptidase inhibitors are isolated that provide a higher inositol (1,4,5) triphosphate concentration than known prolyl endopeptidase inhibitors.
